(19) United States
(12) Patent Application Publication
(10) Pub. No.: US 2007/0269060 A1
(43) Pub. Date: Nov. 22, 2007

(54) Structure and Method of Condenser Microphone Device for Skin-Contact Usage

(75) Inventor: Ching-Tsai Chou, Tai-Chong Hsien (TW)

Correspondence Address: WPAT, PC;INTELLECTUAL PROPERTY ATTORNEYS, 2030 MAIN STREET, SUITE 1300, IRVINE, CA, 92614, US

(73) Assignee: AIRDIGIT INCORPORATION, Hsin-Chu City (TW)

(21) Appl. No.: 11/419,730

(22) Filed: May 22, 2006

(51) Int. Cl. H04R 25/00; H04R 3/00; A61B 7/04

(52) U.S. Cl. 381/174; 381/67; 381/113

(57) ABSTRACT

A number of improved structures and methods to a condenser microphone device are provided so that the condenser microphone device can be used in a skin-contact manner satisfactorily. According to the proposed methods, the sensitivity of the condenser microphone device is lowered appropriately by reducing the aperture of the through hole on the enclosure of the condenser microphone device's sensing member, or by reducing the amount of electrical charge carried by the thin film inside the sensing member. According to the proposed structures, the sensing member is wrapped inside a plastic jacket or film, covering up the through hole on the sensing member's enclosure, or providing another through hole of very small aperture. The plastic jacket could also form a buffer space between the jacket and the sensing member.

### BACKGROUND OF THE INVENTION

1. Field of the Invention

The present invention generally relates to condenser microphone devices, and more particularly to the methods and structures of improving a condenser microphone device for skin-contact usage.

2. The Prior Arts

In general, a conventional condenser, or capacitance-type, microphone device picks up voice from an audio source (such as a speaker, a loud speaker, etc.) via air vibration. However, environmental noise such as those from the whistle of wind, near-by conversation, and a speeding car, is also picked up by the condenser microphone device indiscriminately via the air.

To overcome the problem of using condenser microphone device in a noisy environment, the so-called skin-contact microphone device is provided, which picks up a speaker's voice by sensing the vibration of skin around the speaker's throat. As skin vibration is much stronger than air vibration, the skin-contact microphone device usually utilizes a piezoelectric component such as ceramic to transform the skin vibration into corresponding electrical signal. However, a general piezoelectric microphone device is difficult to pick up high-frequency voice via skin vibration, unless the thickness of a piezoelectric component is reduced to as small as 25 μm. This inevitable makes the manufacturing of the skin-contact piezoelectric microphone device more difficult, resulting in higher production cost. In addition, due to the same issue, a piezoelectric microphone device is usually supplemented with a high-gain amplification circuit which, besides the higher power consumption, makes the piezoelectric microphone device susceptible to noise in the circuit such as the thermal noise.

In contrast, the circuit noise is not a problem to the condenser microphone device, as its sensitivity is much higher than that of the piezoelectric microphone device and, therefore, the auxiliary high-gain amplification circuit is not required. Further more, the condenser microphone device is conventionally simpler and cheaper to manufacture. It is therefore quite natural to consider combining the merits from both sides. However, experiments show that, by using the condenser microphone device in a skin-contact manner in its conventional structure, there are several disadvantages: (1) the voice picked up is distorted or illegible due to the skin vibration being too strong for the conventional condenser microphone device; (2) the environmental noise is inevitable as air vibration cannot be shielded completely; and (3) the condenser microphone device would pick up low-frequency echo (below 500 Hz), which is intensified by the cranium resonance if the microphone device is used to contact facial skin (e.g., when the microphone device is built into a wireless headset for mobile phones).

### SUMMARY OF THE INVENTION

Accordingly, the present invention provides a number of improved structures and methods to a condenser microphone device so that the condenser microphone device can be used in a skin-contact manner satisfactorily. A major aspect of the present invention is that the improved condenser microphone device can pick up a speaker's voice via skin contact clearly and the voice is strong enough to avoid the use of high-gain amplification circuit. The present invention therefore is free from the circuit noise and consumes less power.

Another aspect of the present invention is that, besides the speaker's voice, the improved condenser microphone device can provide a superior shielding effect to the environmental noise. The present invention therefore can be used in a surrounding abundant with deafening noise.

Yet another aspect of the present invention is that the improved condenser microphone device is simple and inexpensive to manufacture, and is immune to the lower-frequency echo and distortion.

The present invention provides several improved structures and methods. In some embodiments of the present invention, the sensitivity of the condenser microphone device is lowered appropriately by reducing the aperture of the through hole on the enclosure of the condenser microphone device's sensing member, or by reducing the amount of electrical charge carried by the thin film inside the sensing member, so that only a fraction of the environmental noise would pass through, or the environmental noise would only produce ignorable electrical signal.

In some other embodiments of the present invention, the sensing member of the improved condenser microphone device is wrapped inside a plastic jacket or film, covering up the through hole on the sensing member's enclosure, or providing another through hole of very small aperture. The configuration of the plastic jacket is to shield the penetration of the sound wave of the environmental noise into the sensing member, and reducing the aperture of the through hole is because the area of the through hole is reciprocal to the volume of environmental noise perceived by the sensing member. In some embodiments, the plastic jacket further forms a buffer space between the jacket and the sensing member. With the configuration of the buffer space, the skin vibration of the speaker compresses the air inside the buffer space first and then is transformed by the sensing member into electrical signal. As such, low-frequency distortion and echo can be effectively reduced.

The improved structures and methods of the present invention can be further augmented by a filtering and amplification means or component, which removes the low-frequency echo and provides a gain that increases along with the frequency of the input electrical signal. The present invention therefore features a superior frequency response.

The foregoing and other objects, features, aspects and advantages of the present invention will become better understood from a careful reading of a detailed description provided herein below with appropriate reference to the accompanying drawings.

### BRIEF DESCRIPTION OF THE DRAWINGS

FIG. 1a is a schematic perspective view of a conventional condenser microphone device.

FIG. 1b is a schematic sectional view of the sensing member of FIG. 1a.

FIG. 1c is a schematic perspective view of the sensing member of FIG. 1a.

FIG. 2a is a schematic sectional view of an embodiment of an improved structure of the condenser microphone device according to the present invention.

FIG. 2b is a schematic sectional view of an embodiment of another improved structure of the condenser microphone device according to the present invention.

FIG. 2c is a schematic sectional view of an embodiment of yet another improved structure of the condenser microphone device according to the present invention.

FIG. 2d is a schematic sectional view of an embodiment of still another improved structure of the condenser microphone device according to the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

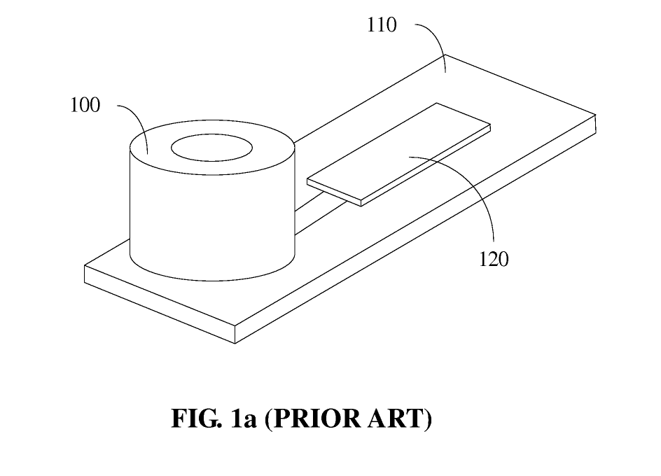

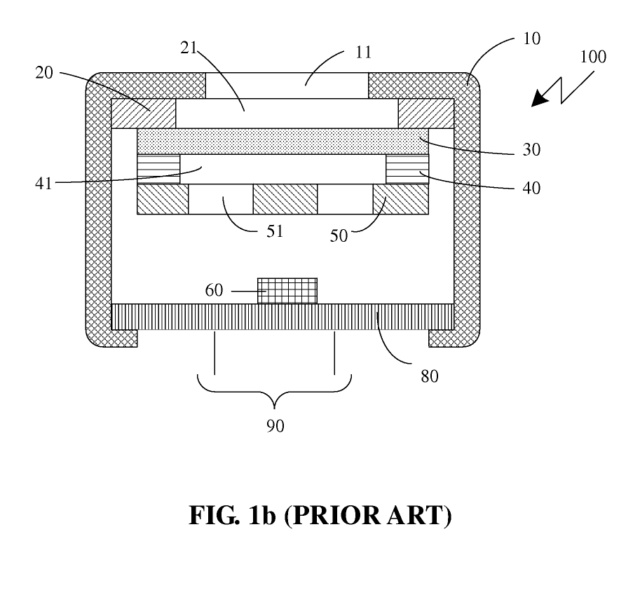

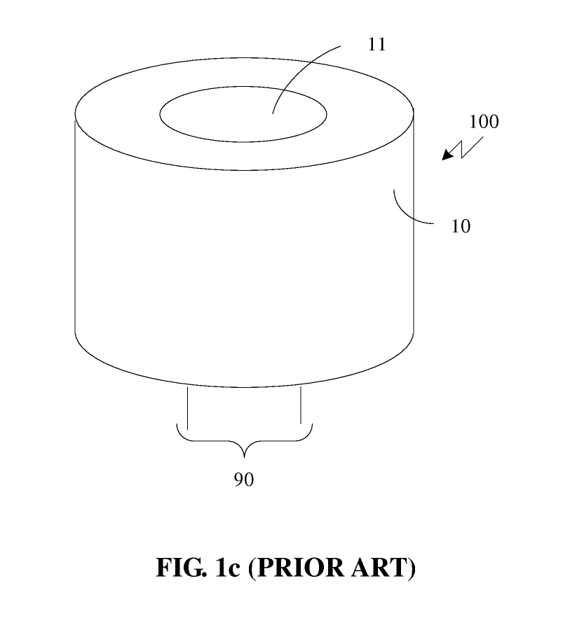

A conventional condenser microphone device, as shown in FIG. 1a, generally contains a sensing member 100, an electronic member 120 usually as part of a circuit board 110, and a casing member (not shown) enclosing the sensing member 100, the electronic member 120, and the circuit board 110 inside.

The improved structures and methods disclosed by the present invention are mainly applied to the sensing member 100 of the microphone device. A microphone device incorporating the present invention is used by having the sensing member (housed in the casing member) in contact with the skin of the speaker, and the skin provides preliminarily shielding to the environmental noise. Then, the improved structures or methods of the present invention further reduce the sensitivity of the sensing member to the environmental noise so as to achieve the required level of noise shielding. The microphone device can be implemented into various applications such as wired or wireless headset of mobile phones. Please note that, the circuit board 110, electronic member 120, and the casing member are omitted from the following description as they are not the subject matter of the present invention. Please also note that the accompanied drawings are not drawn to scale.

FIGS. 1b and 1c provide schematic sectional and perspective views to the sensing member of FIG. 1a. As illustrated, the sensing member 100 has a hollow, cylindrical, metallic enclosure 10. On an end of the enclosure 10, hereinafter referred to as the receiving end, one or more through holes 11 whose aperture is usually at least 2 mm are configured, permitting air vibration to pass through the enclosure 10. The other end of the enclosure 10 is sealed by a circuit board 80. In the following, for ease of reference, the enclosure 10 is used as the base of reference with the receiving end considered at the top, and the circuit board 80 at the bottom.

Right beneath the receiving end, a first metallic plate 20, a charged film 30, an insulating washer 40, and a second metallic plate 50 are arranged in this sequential top-down order. The first metallic plate 20 provides a through hole 21 in the center which is covered entirely by the charged film 30 from below. The through hole 21 allows air to contact the charged film 30 and provides room for the vibration of the charged film 30. The electrical charge carried by the charged film 30 is provided by a polarization process during manufacturing the sensing member 100, and the electrical charge is sealed in silicone or similar material. Also to provide room for the vibration of the charged film 30, a through hole 41 is configured in the center of the washer 40. Similarly, the second metallic plate 50 has a number of through holes 51 to allow air to flow through.

A capacitor is formed by the charged film 30 and the second metallic film 50 with each of them functioning as an electrode to the capacitor. As the second metallic film 50 is induced to carry an equal amount of opposite electrical charge, a potential difference is thereby developed between the two electrodes, which are separated by the washer 40. The potential difference varies along with the change of distance between the two electrodes as the charged film 30 vibrates. An electrical signal corresponding to the vibration of the charged film 30 is therefore obtained. This is the general operation principle of a condenser microphone device.

The electrical signal is then conducted from the electrodes to an impedance transform element 60 on the circuit board 80 via the enclosure 10 and some wiring (not shown). The impedance transform element 60 is usually a field effect transistor (FET) whose main purpose is for impedance matching with the external circuitry outside the sensing member 100. The output of the impedance transform element 60 is provided on the output terminals 90 of the sensing member 100.

When a sensing member as shown in FIGS. 1b and 1c is in contact with the speaker's skin, the skin vibration would compress the air inside the enclosure 10, which in turn engages the charged film 30 into up and down vibration, causing the generation of a corresponding electrical signal. As mentioned earlier, there are a number of shortcomings when the condenser microphone device is used in this manner. First of all, the skin vibration is too strong for an ordinary sensing member 100. Secondly, the environmental noise can still penetrate through the through hole 11 or the wall of the enclosure 10. Additionally, as the through hole 11 is blocked by the skin, the sensing member 100 would pick up low-frequency echo resulted from and intensified by the cranium resonance when the user speaks.

The first method provided by the present invention is to reduce the sensitivity of the sensing member by endowing a smaller amount of electrical charge to the charged film 30 through reducing the voltage and operation time of the polarization process. The sensitivity of a conventional sensing member is around −40 dB, and the sensitivity of a sensing member produced by the present method is reduced to −50˜−60 dB so as to minimize the impact of the environmental noise to the sensing member.

Another method of the present invention is to reduce the aperture or the total area of the through holes 11 from the conventional 2 mm down to less than 1 mm. In this way, only a very small fraction of the air vibration caused by the environmental noise would penetrate into the enclosure 10 via the through holes 11.

To solve the problems of low-frequency distortion from violent skin vibration, of low-frequency echo from the cranium resonance, and of weakened high-frequency signal from the reduction of sensitivity, the foregoing methods are further augmented by a filtering and amplification means. In one way, the electrical signal of the low-frequency distortion and low-frequency echo are filtered and, in another way, the gain of the filtering and amplification means is increased along with the frequency of the electrical signal so as to achieve superior frequency response. In other words, the gain of the filtering and amplification means is smaller at low frequency band and larger at high frequency band within the audible sound frequency range. Even though the sensitivity of the sensing member is reduced through the foregoing methods, it is still relatively sensitive to the skin vibration compared to the conventional piezoelectric sensing members. Therefore, the gain of the filtering and amplification means is moderate and the problem of picking up circuit noise is thereby not significant.

Generally, this filtering and amplification means is implemented by a high-pass amplification circuit. The high-pass amplification circuit takes the output of the sensing member as its input, and produces its output to other circuits connected to the microphone device. The high-pass filter circuit is quite well known to people skilled in the related arts and its details are omitted here.

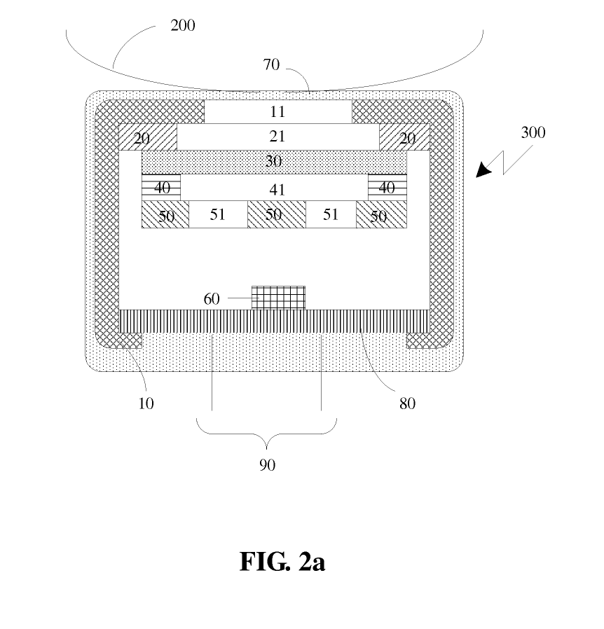

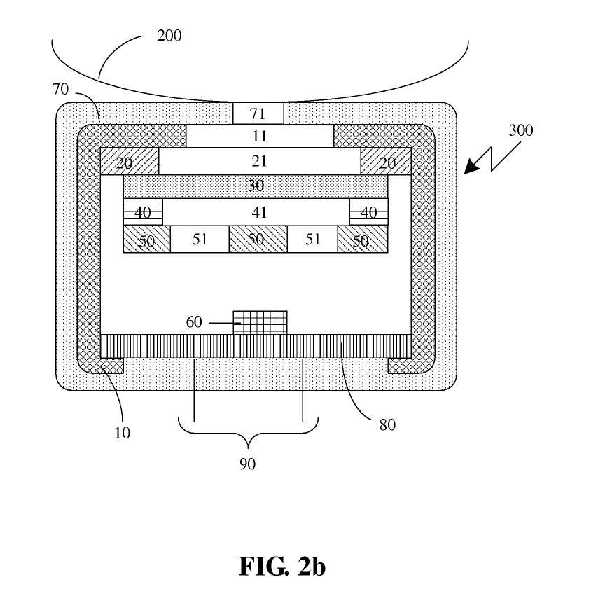

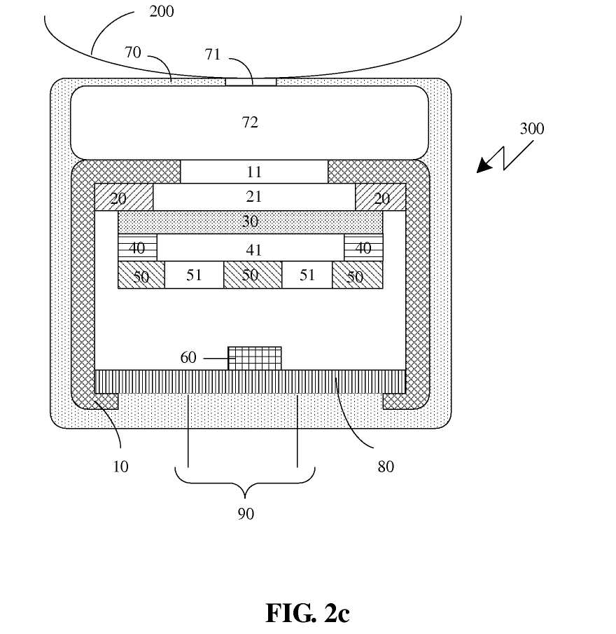

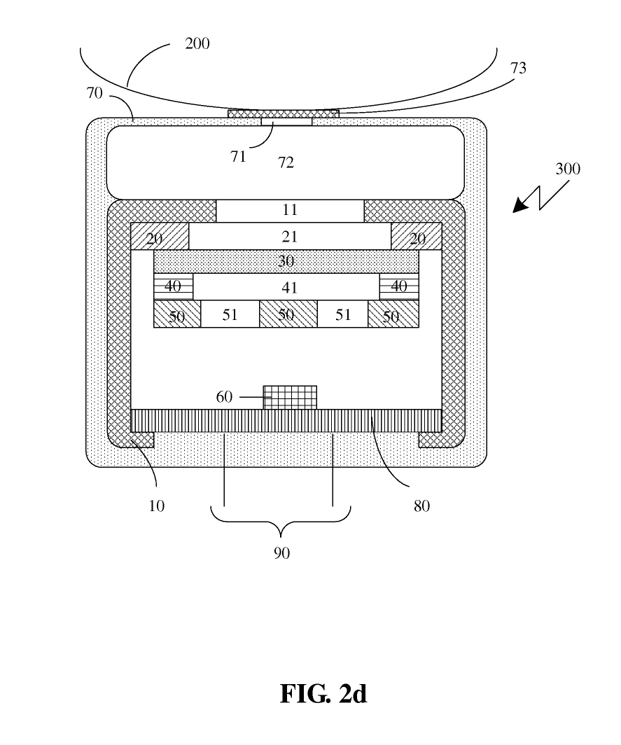

FIGS. 2a˜2c are schematic sectional views of the embodiments of the improved structures of the sensing member according to the present invention. These improved structures all involve the cladding of a jacket or film 70 around the enclosure 10. The jacket 70 is usually made of a flexible material such as plastic, rubber, or artificial rubber, and the jacket 70 covers at least the through hole 11. The purpose of having the plastic jacket 70 is to shield the air vibration from the environmental noise and to reduce its strength in penetrating through the enclosure 10. According to experiments, the jacket 70 can provide a shielding effect between 10˜20 dB. As a result, it is not necessary to reduce the electrical charge of the charged film 30 or the aperture of the through hole 11, and a conventional, inexpensive manufacturing process of the sensing member could be readily adapted for the implementation of a skin-contact microphone device, lowering down the production cost significantly.

As shown in FIG. 2a, the enclosure 10 is entirely sealed by the jacket 70 and, therefore, the jacket 70 has to be rather thin so that it does not offer too strong an attenuation to the sensitivity of the sensing member 300. This inevitably makes the production quite difficult as it is not easy to control the thickness of the jacket 70. Another improved structure shown in FIG. 2b is to provide a through hole 71 on the jacket 70 at a location corresponding to the through hole 11 of the enclosure 10. The through hole 71 has an aperture less than 1 mm, and the aperture is so small that only the stronger air vibration caused by the skin could pass through the through holes 71 and 11 to the inside of the enclosure 10, while most of the weaker air vibration by the environmental noise is blocked. With the configuration of the through hole 71, it is therefore not required to reduce the thickness of the jacket 70, making the production process simpler. Please note that the jacket 70, as shown in FIGS. 2a and 2b, could wrap around the enclosure 10 completely to shield the external air vibration from penetrating the wall of the enclosure 10.

The improved structure shown in FIG. 2c is to reserve a buffer space 72 between the jacket 70 and the receiving end of the enclosure 10. As such, when a user speaks, the vibration of the skin 200 and the air vibration from the environmental noise first compress the air inside the buffer space 72 via the through hole 71. The air vibration inside the buffer space 72 then in turn drives the charged film 30 via the through hole 11. More specifically, the purpose of having such a buffer space 72 is to provide damping to reduce low-frequency distortion.

The improved structure shown in FIG. 2d is very similar to the one shown in FIG. 2c, except that the through hole 71 is further covered by a thin film 73. In this way, the hole 71 is not required to be very small so that ordinary tool can be used to make the hole 71. As the hole 71 has a larger aperture, the thin film 73 is required to prevent air vibration from the environment noise to enter the inside of the enclosure 10.

The foregoing methods and improved structures can be implemented individually, or jointly to achieve even better noise shielding result. For example, the method of reducing the electrical charge of the charged film 30 can be applied along with the configuration of a flexible film or jacket 70 to cover the through hole 11, or along with a flexible film or jacket 70 having a tiny through hole 71, or along with a flexible film or jacket 70 having a buffer space 72 at the receiving end of the enclosure 10.

Similarly, to solve the problems of low-frequency distortion from violent skin vibration, of low-frequency echo from the cranium resonance, and of weakened high-frequency signal from the reduction of sensitivity, the foregoing structure is augmented by a filtering and amplification component that, on one hand, filters the electrical signal of the low-frequency distortion and low-frequency echo and, on the other hand, provides increasing gain along with the frequency of the electrical signal. The filtering and amplification component could be implemented by a high-pass amplification circuit. The high-pass amplification circuit takes the output of the sensing member 300 as its input, and produces its output to other circuits connected to the microphone device.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A method of adapting a condenser microphone device for picking up voice via skin contact, said condenser microphone device comprising a sensing member having a hollow, cylindrical enclosure and a capacitance element, at least a through hole provided on a receiving end of said enclosure, said capacitance element positioned cross-sectionally inside said enclosure at an appropriate distance below said through hole, said capacitance element formed by a thin film and a metallic plate with an appropriate distance therebetween, said method comprising the steps of:
charging said thin film with an appropriate amount of electrical charge so that said sensing member has a sensitivity between −50 dB to −70 dB; and
providing a filtering and amplification means to the electrical signal output from said sensing member, said filtering and amplification means filtering the electrical signal below an appropriate frequency and providing a gain which increases along with the frequency of the electrical signal.

2. A method of adapting a condenser microphone device for picking up voice via skin contact, said condenser microphone device comprising a sensing member having a hollow, cylindrical enclosure and a capacitance element, said capacitance element positioned cross-sectionally at an appropriate location inside said enclosure, said capacitance element formed by a thin film and a metallic plate with an appropriate distance therebetween, said method comprising the steps of:
providing at least a through hole whose aperture is at most 1 mm on a receiving end of said enclosure above said capacitance element; and
providing a filtering and amplification means to the electrical signal output from said sensing member, said filtering and amplification means filtering the electrical signal below an appropriate frequency and providing a gain which increases along with the frequency of the electrical signal.

3. A structure of a condenser microphone device for picking up voice via skin contact, said condenser microphone device having a sensing member and a filtering and amplification component, said sensing member comprising:
a hollow, cylindrical enclosure having a through hole on a receiving end of said enclosure;
a capacitance element, said capacitance element positioned cross-sectionally inside said enclosure at an appropriate distance below said through hole, said capacitance element formed by a charged film and a metallic plate with an appropriate distance therebetween; and
a jacket outside said enclosure covering up at least said through hole;
wherein said filtering and amplification component electrically connected to the output of said sensing member, said filtering and amplification component filtering the electrical signal output from said sensing member below an appropriate frequency and providing a gain which increases along with the frequency of the electrical signal.

4. The structure according to claim 3, wherein said jacket provides a through hole on top of said through hole of said enclosure.

5. The structure according to claim 4, wherein the aperture of said through hole of said jacket is at most 1 mm.

6. The structure according to claim 3, wherein said through hole is covered by a thin film.

7. The structure according to claim 3, wherein said jacket covers the cylindrical wall of said enclosure.

8. The structure according to claim 3, wherein said jacket forms a buffer space between said jacket and said receiving end of said enclosure.

9. The structure according to claim 3, wherein said jacket is made of one of the following material: plastic, rubber, and artificial rubber.
